SEQUENCE LISTING (20 sequences)

SEQ ID NO: 1 - protein, 7 aa, synthetic construct
  VARIANT 5: note = Any amino acid, or an amino acid selected from G, L, M or N. Further, position 5 and position 7 can be the same same or different from each other
  VARIANT 7: note = Any amino acid, or an amino acid selected from G, L, M or N. Further, position 5 and position 7 can be the same same or different from each other
LPPIXRX

SEQ ID NO: 2 - protein, 12 aa, synthetic construct
WDLPPIGRLSGN

SEQ ID NO: 3 - protein, 12 aa, synthetic construct
THLPPIMRNLQF

SEQ ID NO: 4 - skipped

SEQ ID NO: 5 - protein, 4 aa, synthetic construct
GGSK

SEQ ID NO: 6 - protein, 12 aa, synthetic construct
SWMPPILRSPAV

SEQ ID NO: 7 - protein, 12 aa, synthetic construct
SWLPNIQRHWLS

SEQ ID NO: 8 - protein, 12 aa, synthetic construct
HLPPILRMLDLV

SEQ ID NO: 9 - protein, 12 aa, synthetic construct
HLPPIQRTPTYA

SEQ ID NO: 10 - protein, 12 aa, synthetic construct
LPPIVRLPGLLH

SEQ ID NO: 11 - protein, 12 aa, synthetic construct
FPFGPINRDMTA

SEQ ID NO: 12 - protein, 12 aa, synthetic construct
NGVWLPPIARVL

SEQ ID NO: 13 - protein, 5 aa, synthetic construct
GGGSK

SEQ ID NO: 14 - protein, 6 aa, synthetic construct
GGGGSK

SEQ ID NO: 15 - protein, 7 aa, synthetic construct
GGGGGSK

SEQ ID NO: 16 - protein, 7 aa, synthetic construct
  VARIANT 5: note = Any amino acid
  VARIANT 7: note = Any amino acid
LPPIXRX

SEQ ID NO: 17 - protein, 17 aa, synthetic construct
  SITE 17: note = Biotinylated residue
WDLPPIGRLSGNGGGSK

SEQ ID NO: 18 - protein, 17 aa, synthetic construct
  SITE 17: note = Cyanine 5 modified residue
WDLPPIGRLSGNGGGSK

SEQ ID NO: 19 - protein, 4 aa, synthetic construct
GGGS

SEQ ID NO: 20 - protein, 4 aa, synthetic construct
LPPI

(19) United States
(12) Patent Application Publication
(10) Pub. No.: US 2026/0258373 A1
(43) Pub. Date: Sep. 3, 2026

(54) PEPTIDE TAGS FOR PHAGE IMAGING

(71) Applicant: The Regents of the University of California, Oakland, CA (US)

(72) Inventors: Nicole F. Steinmetz, San Diego, CA (US); Robert T. Schooley, San Diego, CA (US); Steffanie A. Strathdee, Carlsbad, CA (US); Soo Khim Chan, Sunnyvale, CA (US)

(21) Appl. No.: 18/881,699

(22) PCT Filed: Jul. 27, 2023
(86) PCT No.: PCT/US2023/028876

§ 371 Date: Jan. 6, 2025

Related U.S. Application Data
(60) Provisional application No. 63/393,744, filed on Jul. 29, 2022.

(51) Int. Cl. C12N 7/00; C07K 7/06; C07K 7/08; C12N 15/10; G01N 33/569

(52) U.S. Cl. CPC C12N 7/00; C07K 7/06; C07K 7/08; C12N 15/1037; G01N 33/56983; C12N 2795/10121

(57) ABSTRACT

Provided herein are isolated peptides that bind to a phage that infects and lyses the pathogenic bacterium P. aeruginosa. The peptides are useful as in vivo tracers for the real-time analysis of phage therapy.

### CROSS-REFERENCE TO RELATED APPLICATION

This application claims priority under 35 U.S.C. § 119(e) to U.S. Provisional Application No. 63/393,744, filed Jul. 29, 2022, the contents of which are incorporated herein by reference in their entireties.

### BACKGROUND

Antimicrobial resistance (AMR) complicates treatment of serious infections with conventional antibiotics. (How Antibiotic Resistance Happens|CDC; accessed Apr. 3, 2022) The World Health Organization (WHO) has declared AMR as one of the top 10 global public health threats, (Antimicrobial resistance https://www.who.int/news-room/fact-sheets/detail/antimicrobial-resistance (accessed Apr. 3, 2022)) with the potential to cause more deaths than major diseases such as HIV and malaria, especially in low-resource settings. (Murray et al., A Systematic Analysis. Lancet, (2022)) In the US alone, more than 2.8 million infections with AMR pathogens are reported every year, leading to more than 35,000 deaths. (About Antibiotic Resistance|CDC https://www.cdc.gov/drugresistance/about.html (accessed Apr. 3, 2022)) AMR is exacerbated by the misuse and overuse of antibiotics in medicine and agriculture, and the lack of novel antibiotics in the drug discovery and development pipeline suggests that the AMR crisis will worsen over time. (Tacconelli et al., Lancet. Infect. Dis., (2018))

Pseudomonas aeruginosa is one of the leading nosocomial AMR pathogens. (Murray et al., A Systematic Analysis. Lancet, (2022)), (Pseudomonas aeruginosa pneumonia—UpToDate https://www.uptodate.com/contents/pseudomonas-aeruginosa-pneumonia (accessed Apr. 21, 2022)) This Gram-negative bacterium is an opportunistic pathogen that rarely infects healthy individuals but is a grave threat to immunocompromised patients. (Laborda et al., Curr. Opin. Microbiol., (2021)). Antimicrobials are the gold standard to treat P. aeruginosa infections, but this creates selective pressure that favors the emergence of AMR strains. (Paterson et al., Clin. Infect. Dis., (2003)) AMR P. aeruginosa can synthesize carbapenemase or Verona integron-encoded metallo-β-lactamase (VIM) to exclude, metabolize or export first-line antibiotics such as amoxicillin, trimethoprim/sulfamethoxazole, or erythromycin, making the infections much harder to treat. (Pseudomonas aeruginosa|HAI|CDC https://www.cdc.gov/hai/outbreaks/pseudomonas-aeruginosa.html (accessed Apr. 21, 2022)), (Spagnolo et al., Rev. Med. Microbiol., (2021)).

Phage therapy is an alternative to conventional antibiotics involving the use of bacteriophages that naturally infect pathogenic bacteria and kill them. (Schooley et al., Nat. Microbiol., (2020)) Interest in phage therapeutics has been rekindled by the AMR crisis. (d'Herelle et al., Bull. N. Y. Acad. Med., (1931)) Like other viruses, phages are abundant and ubiquitous nucleoprotein structures comprising a DNA or RNA genome encased in a proteinaceous capsid. (Brives et al., Palgrave Commun., (2020)) Phage infection is specific to a particular species or even strains of bacteria, killing the cells by lysis or initiating a latent infection known as lysogeny. (Kasman et al., Brenner's Encycl. Genet. Second Ed., (2021)) Phage therapy effectively controls bacterial infections, including P. aeruginosa, (Cafora et al., Cystic Fibrosis Zebrafish Model. Sci. Reports, 2019), (Yang et al., Microbiol., (2021)) and has been used under emergency authorization to save patients infected with multidrug-resistant bacteria. (Novel Phage Therapy Saves Patient with Multidrug-Resistant Bacterial Infection https://health.ucsd.edu/news/releases/pages/2017-04-25-novel-phage-therapy-saves-patient-with-multidrug-resistant-bacterial-infection.aspx (accessed May 9, 2022)); (Aslam et al., Open Forum Infect. Dis., (2020)); (Dedrick et al., Compassionate-Use of Phages in Twenty Patients with Drug-Resistant Mycobacterial Disease. Clin. Infect. Dis. (2022)).

One of the major challenges of phage therapy is the lack of non-invasive methods to determine the phage population in vivo at sites of infection following phage administration. Although phage titers can be determined in blood obtained at various times after intravenous administration by the double layer agar (DLA) technique, phage are cleared from the blood within 90 minutes and have little relationship to concentrations of phage at sites of infection. (Dhungana et al., Microbiol., (2021)); (Shi et al., Microbiol., (2021)) In addition, this is a laborious technique and the results take up to 2 days. (Ács et al., Microbiol., (2020)) Phage labeling with quantum dots, radioisotopes and fluorochromes can be used to monitor the distribution of injected phage, but progeny phage generated following the infection of bacteria cannot be detected. (Kelly et al., Neoplasia, (2006)); (Rusckowski et al., Nucl. Med. Biol., (2008)).

This discloses addresses these limitations of the prior art and provides related advantages as well.

### SUMMARY OF THE DISCLOSURE

Applicant provides herein an isolated peptide comprising, or consisting essentially of, or yet further consisting of the amino acid LPPIX₁RX₂ (SEQ ID NO: 1), wherein X₁ or X₂ is any amino acid, or an amino acid selected from G, L, M or N, and wherein X₁ and X₂ can be the same or different from each other. Non-limiting examples of such include an isolated peptide comprising a peptide selected from the group of peptides shown in FIG. 3, and optionally FIG. 3A or an isolated peptide comprising or consisting essentially of, or yet further consisting of an amino acid selected from the group of: An isolated peptide comprising the amino acid selected from an amino acid from the group of WDLPPIGRLSGN (SEQ ID NO: 2) or THLPPIMRNLQF (SEQ ID NO: 3), SWMPPILRSPAV (SEQ ID NO: 6), SWLPNIQRHWLS (SEQ ID NO: 7), HLPPILRMLDLV (SEQ ID NO: 8), HLPPIQRTPTYA (SEQ ID NO: 9), LPPIVRLPGLLH (SEQ ID NO: 10), FPFGPINRDMTA (SEQ ID NO: 11), or NGVWLPPIARVL (SEQ ID NO: 12). In one aspect, the amino acid is selected from WDLPPIGRLSGN (SEQ ID NO: 2) or THLPPIMRNLQF (SEQ ID NO: 3) or SWMPPILRSPAV (SEQ ID NO: 6).

In one aspect, the isolated peptide further comprises a linker, optionally wherein the linker comprises GGS (SEQ ID NO: 4) or GGSK (SEQ ID NO: 5), or GGGSK (SEQ ID NO: 13). The linker is attached to the N-terminus or the C-terminus of the isolated peptide. In one aspect, the linker is attached to the N-terminus of the isolated peptide.

In another aspect, the isolated peptide further comprises a detectable label, e.g., a fluorescently detectable label selected from biotin or cyanine 5 (Cy5) or a radionucleotide.

This disclosure also provides a plurality of isolated peptides as described herein, that can be the same or different from each other that are optionally detectably labeled.

The isolated peptide of as disclosed herein can be bound to a phage or its progeny, e.g., a Good Vibes phage. Thus, in one aspect, provided herein is a conjugate wherein the isolated peptide of this disclosure is bound to a phage or its progeny, e.g., a Good Vibes phage.

Further provided are polynucleotides encoding the isolated peptides of this disclosure, wherein the polynucleotide is DNA or RNA, or a complement of each thereof. Also provided are vectors or host cells comprising one or more of the isolated peptide, or the polynucleotide of this disclosure. The vector can further comprise one or more of a promoter or an enhancer, and further optionally wherein the vector is a viral vector or a plasmid. Further provided is an isolated host cell comprising one or more of the isolated peptide, the plurality of the isolated peptides, the conjugate or the polynucleotide of this disclosure. The host cell can be a prokaryotic or a eukaryotic cell.

Compositions are also provided. The composition comprises a carrier and one or more of: the isolated peptide, the plurality of the isolated peptides, the polynucleotide, the vector or host cell of this disclosure. In one aspect, the composition further comprises a stabilizer or preservative. In one aspect, the stabilizer or preservative is non-naturally occurring.

The peptides and compositions are useful to detect a phage in a sample, by contacting the sample with an isolated peptide or a plurality of isolated peptides of this disclosure and detecting the binding of the peptide to the phage, wherein the presence of binding is a detection of the phage in the sample. The sample can be a clinical isolate or other sample where phages may be present.

The peptides and compositions are useful to detect cells infected with the GV phage, such as P. aeruginosa cells by contacting the sample with an isolated peptide or a plurality of isolated peptides of this disclosure and detecting the binding of the peptide to the phage in the cell, wherein the presence of binding is a detection of the cell containing the phage in the sample. The sample can be a clinical isolate or other sample where infected cells may be present. Methods of detection will vary with the label, and such methods are known in the art.

The peptides and compositions are further useful to label a phage in a sample, comprising contacting the sample with an isolated peptide or plurality of peptides as described herein, and detecting the binding of the peptide to the phage, wherein the presence of binding is a detection of the phage in the sample. The sample can be a clinical isolate or other sample where phages may be present. Methods of detection will vary with the label, and such methods are known in the art.

The isolated peptides also are useful to monitor anti-pseudomonas therapy by administering to a subject in need thereof a radiolabeled isolated peptide of this disclosure to a subject in need thereof and detecting the presence of any peptide bound to a GV phage or GV phage infected cell if present in the subject. Any method to detect radiolabels in a subject can be used in combination with this method. In one aspect, the anti-pseudomonas therapy comprises a therapy to treat an GV phage infected cell. Methods of detection will vary with the label, and such methods are known in the art. The subject can be a mammal, such as a human patient.

### BRIEF DESCRIPTION OF THE FIGURES

FIGS. 1A-1C: Characterization of Good Vibes phage (GV). (FIG. 1A) Transmission electron micrograph of negatively stained GV. (FIG. 1B) Spot test assay of GV using the double layer agar technique. (FIG. 1C) Complete genome map of GV visualized using SnapGene Viewer.

FIGS. 2A-2B: Detection of GV-binding monoclonal phages by ELISA. (FIG. 2A) Polyclonal ELISA of enriched binders from each round against GV. (FIG. 2B) Monoclonal ELISA of 40 monoclonal phages against GV (dots) and BSA (negative control, squares).

FIGS. 3A-3D: Analysis of the monoclonal phages that bind GV. (FIG. 3A) Sequences of GV-binding peptides with a highly conserved motif shown bolded. (FIG. 3B) Heat map showing the cross-reactivity of GV monoclonal phages against GV (target), MAT (same family as GV), cowpea mosaic virus (CPMV, unrelated plant virus), and bovine serum albumin (BSA). (FIG. 3C) Sequence alignment of all peptides from GV monoclonal phages using the T-coffee multiple sequence alignment server (https://tcoffee.crg.eu/, last accessed on Jul. 24, 2023). (FIG. 3D) SAROTUP analysis (http://i.uestc.edu.cn/sarotup/cgi-bin/TUPScan.pl, last accessed on Jul. 24, 2023) of the potential GV-binding peptide WDLPPIGRLSGN. Number in bracket represents the probability in percentage.

FIGS. 4A-4B: Analysis of GVER2738 binding activity by ELISA. (FIG. 4A) The binding of monoclonal phage GVER2738 to the target GV, the closely related virus MAT, the unrelated virus GVTRI-180 (Siphoviridae), and BSA (negative control), as determined by ELISA. (FIG. 4B) The binding of monoclonal phage GVER2738 and the empty phage M13KE to GV and MAT. Data are means±standard deviations (n=3, one-way ANOVA; **p<0.01).

FIGS. 5A-5C: Analysis of GVER2738 binding activity by competitive ELISA. (FIG. 5A) Competitive ELISA between GVER2738 and GVER2738-SYBR against GV (target) and GVBSA (negative control). Agarose gel shows the intercalation of SYBR with GV under UV light. The same gel was stained with Coomassie Brilliant Blue to show the colocalization of phage nucleic acid and coat proteins. (FIG. 5B) ELISA showing the binding of GVBP-biotin to GV and (FIG. 5C) GVBP-Cy5 to GV. Data are means±standard deviations (n=3, one-way ANOVA; *p<0.05).

FIGS. 6A-6B: Analysis of GVBP-Cy5 binding by flow cytometry. (FIG. 6A) Scatter plots showing percent of APC+ cells by gating on P. aeruginosa cells. Mean APC+ population is shown as an inset. (FIG. 6B) Histogram showing median fluorescence intensity (MFI) values of APC+ cells. Data are means±standard deviations (n=3, one-way ANOVA; ****p<0.0001).

### DETAILED DESCRIPTION OF THE DISCLOSURE

### Definitions

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the disclosure to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the disclosure. All publications, patent applications, patents and other references mentioned herein are incorporated by reference in their entirety.

Unless the context indicates otherwise, it is specifically intended that the various features of the disclosure described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

Throughout this disclosure, various publications, patents and published patent specifications may be referenced by an identifying citation or by an Arabic numeral or first author name. The full citation for the publications identified by an Arabic numeral or first author name are found immediately preceding the claims. The disclosures of these publications, patents and published patent specifications are hereby incorporated by reference into the present disclosure in their entirety to more fully describe the state of the art to which this disclosure pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the description of the disclosure and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Embodiments defined by each of these transition terms are within the scope of this disclosure

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

“Eukaryotic cells” comprise, or alternatively consist essentially of, or yet further consist of all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human,

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

A “composition” typically intends a combination of the active agent, e.g., the nanoparticle of this disclosure and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

As used herein, the terms “nucleic acid sequence” and “polynucleotide” and “oligonucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “isolated cell” generally refers to a cell that is substantially separated from other cells of a tissue. The term includes prokaryotic and eukaryotic cells.

As used herein, the term “vector” refers to a nucleic acid construct deigned for transfer between different hosts, including but not limited to a plasmid, a virus, a cosmid, a phage, a BAC, a YAC, etc. A “viral vector” is defined as a recombinantly produced virus or viral particle that comprises a polynucleotide to be delivered into a host cell, either in vivo, ex vivo or in vitro. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Further details as to modern methods of vectors for use in gene transfer may be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif) and Promega Biotech (Madison, Wis.).

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical,” percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

It is to be inferred without explicit recitation and unless otherwise intended, that when the present disclosure relates to a polypeptide, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide or nucleic acid, intends those having minimal homology while still maintaining desired structure or functionality. Unless specifically recited herein, it is contemplated that any of the above also includes equivalents thereof. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and/or exhibits substantially equivalent biological activity to the reference protein, polypeptide, or nucleic acid. Alternatively, when referring to polynucleotides, an equivalent thereof is a polynucleotide that hybridizes under stringent conditions to the reference polynucleotide or its complement.

The phrase “equivalent polypeptide” or “equivalent peptide fragment” refers to protein, polynucleotide, or peptide fragment encoded by a polynucleotide that hybridizes to a polynucleotide encoding the exemplified polypeptide or its complement of the polynucleotide encoding the exemplified polypeptide, under high stringency and/or which exhibit similar biological activity in vivo, e.g., approximately 100%, or alternatively, over 90% or alternatively over 85% or alternatively over 70%, as compared to the standard or control biological activity. Additional embodiments within the scope of this disclosure are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology. Percentage homology can be determined by sequence comparison using programs such as BLAST run under appropriate conditions. In one aspect, the program is run under default parameters.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. A high stringency hybridization refers to a condition in which hybridization of an oligonucleotide to a target sequence comprises no mismatches (or perfect complementarity). Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

As used herein, the term “enhancer”, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

The term “introduce” as applied to methods of producing modified cells refers to the process whereby a foreign (i.e. extrinsic or extracellular) agent is introduced into a host cell thereby producing a cell comprising the foreign agent. Methods of introducing nucleic acids include but are not limited to transduction, retroviral gene transfer, transfection, electroporation, transformation, viral infection, and other recombinant DNA techniques known in the art. In some embodiments, transduction is done via a vector (e.g., a viral vector). In some embodiments, transfection is done via a chemical carrier, DNA/liposome complex, or micelle (e.g., Lipofectamine (Invitrogen)). In some embodiments, viral infection is done via infecting the cells with a viral particle comprising the polynucleotide of interest.

The term “culturing” refers to growing cells in a culture medium under conditions that favor expansion and proliferation of the cell. The term “culture medium” or “medium” is recognized in the art and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium.”

A “gene” refers to a polynucleotide containing at least one open reading frame (ORF) that is capable of encoding a particular polypeptide or protein after being transcribed and translated.

The term “express” refers to the production of a gene product.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

A “gene product” or alternatively a “gene expression product” refers to the amino acid (e.g., peptide or polypeptide) generated when a gene is transcribed and translated.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell. In one aspect, this invention provides promoters operatively linked to the downstream sequences, e.g., suicide gene, VEGF, 165A VEGF, tet activator, etc.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

A “probe” when used in the context of polynucleotide manipulation refers to an oligonucleotide that is provided as a reagent to detect a target potentially present in a sample of interest by hybridizing with the target. Usually, a probe will comprise a detectable label or a means by which a label can be attached, either before or subsequent to the hybridization reaction. Alternatively, a “probe” can be a biological compound such as a polypeptide, antibody, or fragments thereof that is capable of binding to the target potentially present in a sample of interest.

“Detectable labels”, “labels” or “markers” include, but are not limited to radioisotopes, fluorochromes, chemiluminescent compounds, dyes, and proteins, including enzymes. Detectable labels can also be attached to a polynucleotide, polypeptide, antibody or composition described herein. “Detectable label” or “label” as used herein refers to a molecule or a compound or a group of molecules or a group of compounds used to target of interest. In some cases, the detectable label can be detected directly. In other cases, the detectable label can be a part of a binding pair, which can then be subsequently detected. Signals from the detectable label can be detected by various means and will depend on the nature of the detectable label. Detectable labels can be isotopes, fluorescent moieties, colored substances, and the like. Examples of means to detect detectable label include but are not limited to spectroscopic, photochemical, biochemical, immunochemical, electromagnetic, radiochemical, or chemical means, such as fluorescence, chemifluorescence, or chemiluminescence, or any other appropriate means. Non-limiting examples include fluorescent labels such as Cy5 (see https://broadpharm.com/product-categories/fluorescent-dye/cy5-labeling?gad=1&gclid=EAIaIQobChMI-eKsm4-ogAMVwSvUAR0nIgT5EAAYASAAEgJKtPD_BwE, last access on Jul. 24, 2023),

Non-limiting exemplary detectable labels also include a radioactive material, such as a radioisotope, a metal or a metal oxide. Radioisotopes include radionuclides emitting alpha, beta or gamma radiation. In particular embodiments, a radioisotope can be one or more of: ³H, ¹⁰B, ¹⁸F, ¹¹C, ¹⁴C, ¹³N, ¹⁸O, ¹⁵O, ³²P, P³³, ³⁵S, ³⁵Cl, ⁴⁵Ti, ⁴⁶Sc, ⁴⁷Sc, ⁵¹Cr, ⁵²Fe, ⁵⁹Fe, ⁵⁷Co, ⁶⁰Cu, ⁶¹Cu, ⁶²Cu, ⁶⁴Cu, ⁶⁷Cu, ⁶⁷Ga, ⁶⁸Ga, ⁷²As, ⁷⁶Br, ⁷⁷Br, ^{81m}Kr, ⁸²Rb, ⁸⁵Sr, ⁸⁹Sr, ⁸⁶Y, ⁹⁰Y, ⁹⁵Nb, ^{94m}Tc, ^{99m}Tc, ⁹⁷Ru, ¹⁰³Ru, ¹⁰⁵Rh, ¹⁰⁹Cd, ¹¹¹In, ¹¹³Sn, ¹¹³In, ¹¹⁴In, I¹²⁵, I¹³¹, ¹⁴⁰La, ¹⁴¹Ce, ¹⁴⁹Pm, ¹⁵³Gd, ¹⁵⁷Gd, ¹⁵³Sm, ¹⁶¹Tb, ¹⁶⁶Dy, ¹⁶⁶Ho, ¹⁶⁹Er, ¹⁶⁹Y, ¹⁷⁵Yb, ¹⁷⁷Lu, ¹⁸⁶Re, ¹⁸⁸Re, ²⁰¹Tl, ²⁰³Pb, ²¹¹At, ²¹²Bi or ²²⁵Ac.

Additional non-limiting exemplary detectable labels include a metal or a metal oxide. In particular embodiments, a metal or metal oxide is one or more of: gold, silver, copper, boron, manganese, gadolinium, iron, chromium, barium, europium, erbium, praseodynium, indium, or technetium. In additional embodiments, a metal oxide includes one or more of: Gd(III), Mn(II), Mn(III), Cr(II), Cr(III), Cu(II), Ffe (III), Pr(III), Nd(III) Sm(III), Tb(III), Yb(III) Dy(III), Ho(III), Eu(II), Eu(III), or Er(III).

Further non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); or a bioluminescent material (e.g., luciferase, luciferin, aequorin).

Additional non-limiting examples of tags and/or detectable labels include enzymes (horseradish peroxidase, urease, catalase, alkaline phosphatase, beta-galactosidase, chloramphenicol transferase); enzyme substrates; ligands (e.g., biotin); receptors (avidin); GST-, T7-, His-, myc-, HA- and FLAG®-tags; electron-dense reagents; energy transfer molecules; paramagnetic labels; fluorophores (Cy5, fluorescein, fluorscamine, rhodamine, phycoerthrin, phycocyanin, allophycocyanin); chromophores; chemi-luminescent (imidazole, luciferase, acridinium, oxalate); and bio-luminescent agents.

As set forth herein, a detectable label or tag can be linked or conjugated (e.g., covalently) isolated peptide. In various embodiments a detectable label, such as a fluorescent label is bound or conjugated to the agent, either directly or indirectly. A linker or an intermediary functional group can be used to link the molecule to a detectable label or tag. Linkers include amino acid or peptidomimetic sequences inserted between the molecule and a label or tag so that the two entities maintain, at least in part, a distinct function or activity. Linkers may have one or more properties that include a flexible conformation, an inability to form an ordered secondary structure or a hydrophobic or charged character which could promote or interact with either domain. Amino acids typically found in flexible protein regions include Gly, Asn and Ser. The length of the linker sequence may vary without significantly affecting a function or activity. Non-limiting examples of such include GGS (SEQ ID NO: 4) or GGSK (SEQ ID NO: 5), GGGSK, GGGGSK, or GGGGGSK.

Linkers further include chemical moieties, conjugating agents, and intermediary functional groups. Examples include moieties that react with free or semi-free amines, oxygen, sulfur, hydroxy or carboxy groups. Such functional groups therefore include mono and bifunctional crosslinkers, such as sulfo-succinimidyl derivatives (sulfo-SMCC, sulfo-SMPB), in particular, disuccinimidyl suberate (DSS), BS3 (Sulfo-DSS), disuccinimidyl glutarate (DSG) and disuccinimidyl tartrate (DST). Non-limiting examples include diethylenetriaminepentaacetic acid (DTPA) and ethylene diaminetetracetic acid.

As used herein, the term “vector” refers to a non-chromosomal nucleic acid comprising an intact replicon such that the vector may be replicated when placed within a cell, for example by a process of transformation. Vectors may be viral or non-viral. Viral vectors include retroviruses, adenoviruses, herpesvirus, bacculoviruses, modified bacculoviruses, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

In aspects where gene transfer is mediated by a lentiviral vector, a vector construct refers to the polynucleotide comprising the lentiviral genome or part thereof, and a therapeutic gene. As used herein, “lentiviral mediated gene transfer” or “lentiviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, lentiviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral vectors, New York: Spring-Verlag Berlin Heidelberg.

Lentiviral vectors of this invention are based on or derived from oncoretroviruses (the sub-group of retroviruses containing MLV), and lentiviruses (the sub-group of retroviruses containing HIV). Examples include ASLV, SNV and RSV all of which have been split into packaging and vector components for lentiviral vector particle production systems. The lentiviral vector particle according to the invention may be based on a genetically or otherwise (e.g. by specific choice of packaging cell system) altered version of a particular retrovirus.

That the vector particle according to the invention is “based on” a particular retrovirus means that the vector is derived from that particular retrovirus. The genome of the vector particle comprises components from that retrovirus as a backbone. The vector particle contains essential vector components compatible with the RNA genome, including reverse transcription and integration systems. Usually these will include gag and pol proteins derived from the particular retrovirus. Thus, the majority of the structural components of the vector particle will normally be derived from that retrovirus, although they may have been altered genetically or otherwise so as to provide desired useful properties. However, certain structural components and in particular the env proteins, may originate from a different virus. The vector host range and cell types infected or transduced can be altered by using different env genes in the vector particle production system to give the vector particle a different specificity.

The term “promoter” refers to a region of DNA that initiates transcription of a particular gene. The promoter includes the core promoter, which is the minimal portion of the promoter required to properly initiate transcription and can also include regulatory elements such as transcription factor binding sites. The regulatory elements may promote transcription or inhibit transcription. Regulatory elements in the promoter can be binding sites for transcriptional activators or transcriptional repressors. A promoter can be constitutive or inducible. A constitutive promoter refers to one that is always active and/or constantly directs transcription of a gene above a basal level of transcription. Non-limiting examples of such include the phosphoglycerate kinase 1 (PGK) promoter; SSFV, CMV, MNDU3, SV40, Ef1a, UBC and CAGG. An inducible promoter is one which is capable of being induced by a molecule or a factor added to the cell or expressed in the cell. An inducible promoter may still produce a basal level of transcription in the absence of induction, but induction typically leads to significantly more production of the protein. Promoters can also be tissue specific. A tissue specific promoter allows for the production of a protein in a certain population of cells that have the appropriate transcriptional factors to activate the promoter.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

A “composition” is intended to mean a combination of active polypeptide, polynucleotide or antibody and another compound or composition, inert (e.g. a detectable label) or active (e.g. a gene delivery vehicle).

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present invention is also useful for veterinary treatment of companion mammals, exotic animals and domesticated animals, including mammals, rodents, and the like which is susceptible to RNA and in particular, HIV viral infection. In one embodiment, the mammals include horses, dogs, and cats. In another embodiment of the present invention, the human is an adolescent or infant under the age of eighteen years of age.

“Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, a biological sample, or a sample, is obtained from a subject. Exemplary samples include, but are not limited to, cell sample, tissue sample, tumor biopsy, liquid samples such as blood and other liquid samples of biological origin (including, but not limited to, ocular fluids (aqueous and vitreous humor), peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood.

A “pharmaceutical cryoprotectants” are known in the art and include without limitation, e.g., sucrose, trehalose, and glycerol. A cryoprotectant exhibiting low toxicity in biological systems is generally used.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include intraperitoneal administration, and oral administration.

### Modes for Carrying Out the Disclosure

In one embodiment, Applicant provides herein an alternative approach to label phage by introducing a fluorophore-conjugated peptide that binds non-covalently to the phage surface. Applicant identified peptides that bind specifically to the Good Vibes phage (GV), which infects P. aeruginosa. The peptides were isolated from a phage display library by three rounds of biopanning. Monoclonal library phages featured a highly conserved consensus motif (LPPIXRX) in the peptide sequences. The corresponding peptide was synthesized, labeled with biotin or cyanine 5 (Cy5); the binding specificity of the peptide for GV was evaluated by enzyme-linked immunosorbent assay (ELISA). Finally, Applicant used flow cytometry to confirm that the Cy5-labeled peptide preferentially bound to GV attached to the surface of P. aeruginosa cells.

Thus, in one aspect, provided herein are novel reagents that can tag phages for imaging and quantification at clinically relevant locations at sites of infection. Isolated monoclonal library phages featured a highly conserved consensus motif, LPPIXRX. The corresponding peptide WDLPPIGRLSGN was synthesized with a GGGSK linker and conjugated to cyanine 5 or biotin. The specific binding of the LPPIXRX motif to GV in vitro was confirmed using an enzyme-linked immunosorbent assay. It was demonstrated using imaging and tracking of GV in bacterial populations with fluorescent targeting peptide and flow cytometry. In conclusion, Applicant developed fluorescent labeled peptides that bind to bacteriophage GV specifically, which can enable real-time analysis of phage in vivo and monitor the efficacy of phage therapy.

The peptide-tag enables imaging and tracking of the phages can be used to provide information about clinical outcomes. Phages are live therapeutics, therefore imaging of phage amplification in real time would provide not only information about the pharmacodynamics, but also allow a physician to image whether or not is replicating or not; replication would indicate that the phages are lysing the bacteria—meaning success in treatment. Having this information in real-time would allow to make clinical decisions faster and save lives.

Additionally, the peptide-tag allows for additional functionalization; for example a chemotherapy or antibiotic or immunostimulatory agent can be loaded on the phage for targeted delivery to the site of infection. This would allow for any combination of therapeutics to be co-delivered with the therapeutic phage and increase tissue specificity and therefore therapeutic outcomes.

In addition to loading therapeutic molecules via the peptide; targeting ligands can be loaded to direct the phages to specific cells and tissues. Phage therapy is a powerful and emerging technology, clinical trials for phage therapy are just beginning with P. aeruginosa as a main target for many of them. There is an urgent need for pharmacodynamic investigation, but imaging technologies that allow the dynamic, real-time imaging of infectious bacteria combat are lacking. Radiolabeled phages for in vivo tracking have been reported; however, the direct labeling of phage is technologically cumbersome, may alter the pharmacodynamics, host specificity, and ultimately therapeutic potency, and most critically, only allows monitoring the first pass administered dose. It should be noted that unlike conventional antibiotics, phage replicate inside the bacterial host and they produce progeny virions as long as susceptible host cells exist. Therefore, insight into the location and mobility of progeny phage, their replication, persistence, and clearance is required.

The present disclosure is one of a kind solution that allows one to image first pass and progeny phage—and therefore the disclosure would direct and immediate application in these trials to inform outcomes related to PK and efficacy. The technology enables the imaging of the first pass and progeny phage. To the best of Applicant's knowledge, there is no competing technology. Ultimately, this disclosure would provide information about treatment response and disease progression in real time; the clinical impact thus is tremendous in particular for life-threatening infections where time is of the essence.

### Modes for Carrying Out the Disclosure

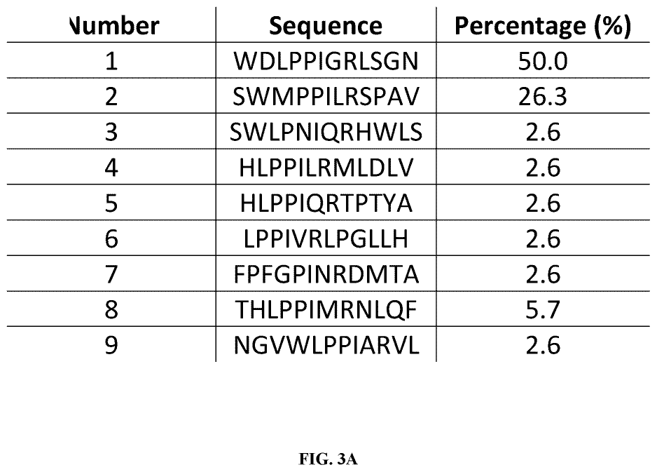

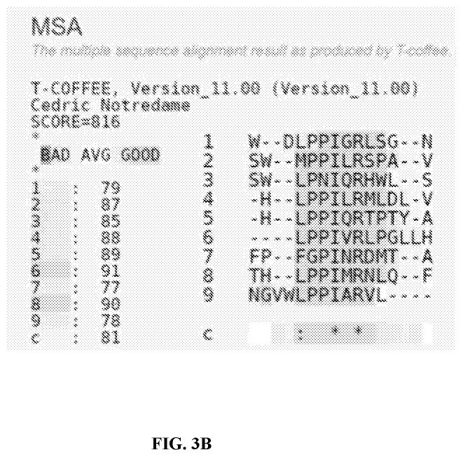

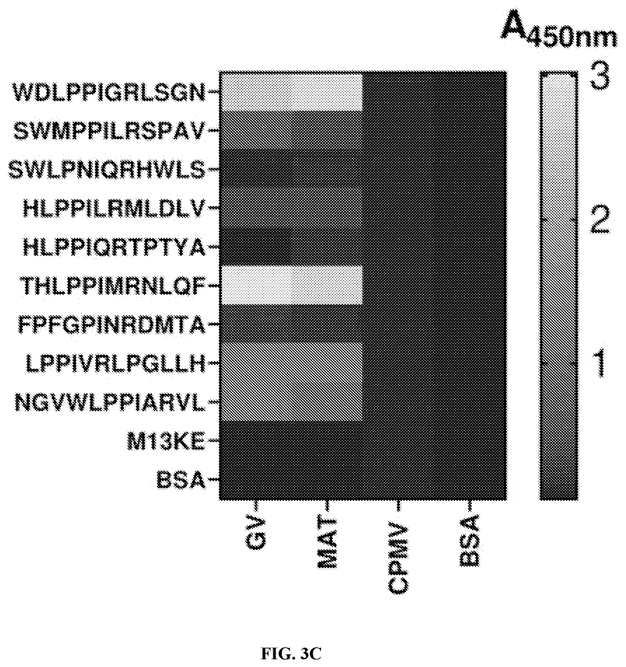

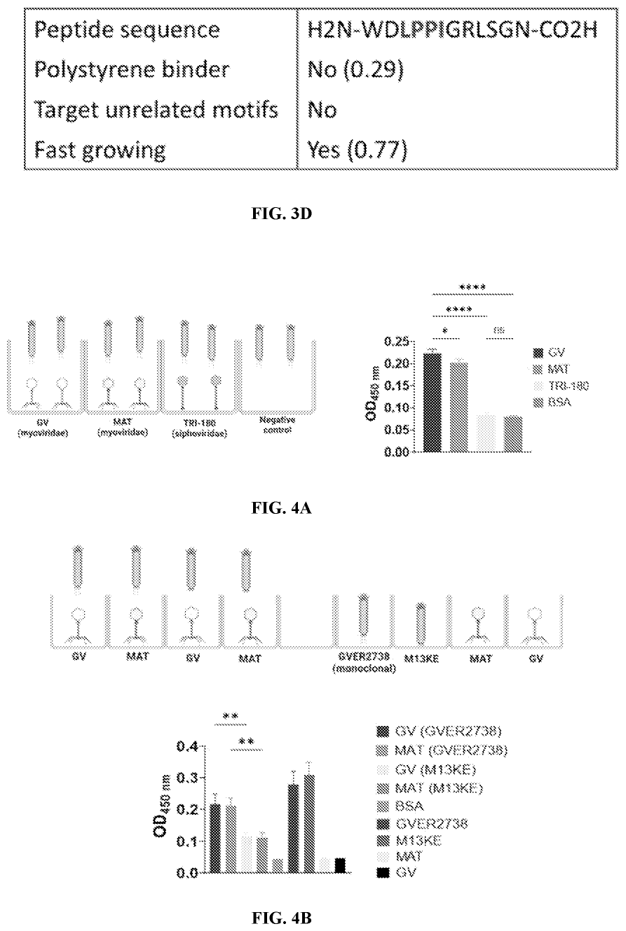

An isolated peptide is provided, the isolated peptide comprising, or consisting essentially of, or yet further consisting of the amino acid LPPIX₁RX₂ (SEQ ID NO: 1), wherein X₁ or X₂ is any amino acid, or an amino acid selected from G, L, M or N, and wherein X₁ and X₂ can be the same or different from each other. Non-limiting examples of such include an isolated peptide comprising a peptide selected from the group of peptides shown in FIG. 3, and optionally FIG. 3A or an isolated peptide comprising or consisting essentially of, or yet further consisting of the amino acid selected from WDLPPIGRLSGN (SEQ ID NO: 2) or THLPPIMRNLQF (SEQ ID NO: 3), SWMPPILRSPAV, SWLPNIQRHWLS, HLPPILRMLDLV, HLPPIQRTPTYA, LPPIVRLPGLLH, FPFGPINRDMTA, or NGVWLPPIARVL. In a further aspect, the isolated peptide comprises, or consists essentially of, or yet further consists of a peptide that comprises, or consists essentially of, or yet further consist of the amino acid WDLPPIGRLSGN (SEQ ID NO: 2).

Also provided is an isolated peptide is provided, the isolated peptide comprising, or consisting essentially of, or yet further consisting of LPPIX₁RX₂ (SEQ ID NO: 1), wherein X₁ or X₂ is any amino acid, or an amino acid selected from G, L, M or N, and wherein X₁ and X₂ can be the same or different from each other. Non-limiting examples of such include an isolated peptide comprising a peptide selected from the group of peptides shown in FIG. 3, and optionally FIG. 3A or an isolated peptide comprising or consisting essentially of, or yet further consisting of the amino acid selected from WDLPPIGRLSGN (SEQ ID NO: 2) or THLPPIMRNLQF (SEQ ID NO: 3), SWMPPILRSPAV, SWLPNIQRHWLS, HLPPILRMLDLV, HLPPIQRTPTYA, LPPIVRLPGLLH, FPFGPINRDMTA, or NGVWLPPIARVL joined to a peptide linker, e.g., a linker selected from GGS (SEQ ID NO: 4) or GGSK (SEQ ID NO: 5) or GGGSK. Also provided is an isolated peptide of the amino acid WDLPPIGRLSGN (SEQ ID NO: 2) that is joined to a peptide linker, e.g., a linker selected from GGS (SEQ ID NO: 4) or GGSK (SEQ ID NO: 5) or GGGSK (SEQ ID NO: 13).

In one aspect, the isolated peptide is a labeled, e.g., detectably labeled isolated peptide comprising, or consisting essentially of, or yet further consisting of the amino acid LPPIX₁RX₂ (SEQ ID NO: 1), wherein X₁ or X₂ is any amino acid, or an amino acid selected from G, L, M or N, and wherein X₁ and X₂ can be the same or different from each other. Non-limiting examples of such include an isolated peptide comprising a peptide selected from the group of peptides shown in FIG. 3, and optionally FIG. 3A or an isolated peptide comprising or consisting essentially of, or yet further consisting of the amino acid selected from WDLPPIGRLSGN (SEQ ID NO: 2) or THLPPIMRNLQF (SEQ ID NO: 3), SWMPPILRSPAV, SWLPNIQRHWLS, HLPPILRMLDLV, HLPPIQRTPTYA, LPPIVRLPGLLH, FPFGPINRDMTA, or NGVWLPPIARVL. In a further aspect, the isolated peptide comprises, or consists essentially of, or yet further consists of the amino acid WDLPPIGRLSGN (SEQ ID NO: 2).

In one aspect, also provided is an isolated peptide that is a labeled, e.g., detectably labeled isolated peptide comprising, or consisting essentially of, or yet further consisting of the amino acid LPPIX₁RX₂ (SEQ ID NO: 1), wherein X₁ or X₂ is any amino acid, or an amino acid selected from G, L, M or N, and wherein X₁ and X₂ can be the same or different from each other. Non-limiting examples of such include an isolated peptide comprising a peptide selected from the group of peptides shown in FIG. 3, and optionally FIG. 3A or an isolated peptide comprising or consisting essentially of, or yet further consisting of the amino acid selected from WDLPPIGRLSGN (SEQ ID NO: 2) or THLPPIMRNLQF (SEQ ID NO: 3), SWMPPILRSPAV (SEQ ID NO: 6), SWLPNIQRHWLS (SEQ ID NO: 7), HLPPILRMLDLV (SEQ ID NO: 8), HLPPIQRTPTYA (SEQ ID NO: 9), LPPIVRLPGLLH (SEQ ID NO: 10), FPFGPINRDMTA (SEQ ID NO: 11), or NGVWLPPIARVL (SEQ ID NO: 12) joined to a peptide linker, e.g., a linker selected from GGS (SEQ ID NO: 4) or GGSK (SEQ ID NO: 5) or GGGSK (SEQ ID NO: 13). Also provided is labeled isolated peptide comprising or consisting essentially of, or yet consisting of the amino acid WDLPPIGRLSGN (SEQ ID NO: 2) that is joined to a peptide linker, e.g., a linker selected from GGS (SEQ ID NO: 4) or GGSK (SEQ ID NO: 5) or GGGSK (SEQ ID NO: 13).

This disclosure also provides a plurality of isolated peptides as described herein, that can be the same or different from each other.

Further disclosed is an isolated peptide as disclosed herein bound to a phage or its progeny, e.g., a Good Vibes phage.

Further provided are polynucleotides encoding the isolated peptide of this disclosure, wherein the polynucleotide is DNA or RNA. Also provided are vectors or host cells comprising one or more of the isolated peptide, or the polynucleotide of this disclosure. The vector can further comprise one or more of a promoter or an enhancer, and further optionally wherein the vector is a viral vector or a plasmid. The polynucleotides, vectors, or host cell are useful to recombinantly reproduce the isolated peptides as disclosed herein.

Compositions are also provided. The composition comprises a carrier and one or more of: the isolated peptide, the polynucleotide, the vector or host cell of this disclosure. In one aspect, the composition further comprises a stabilizer or preservative, and optionally wherein the host cell is a prokaryotic cell or a eukaryotic cell. In one aspect, the compositions further contain a lyophilization agent that permits storage and transport of the peptides.

The peptides and compositions are useful to detect a phage (e.g., a GV phage) or cell infected with the phage in a sample, the method comprising, or consisting essentially of, or yet further consisting of contacting the sample with the peptide of this disclosure or the plurality of peptides, and detecting the binding of the peptide to the phage, wherein the presence of binding is a detection of the phage in the sample. The sample can be a clinical isolate or other sample where phages may be present. The sample can be from any species or subject, e.g. a mammal such as a human patient.

In one aspect of the above method, the isolated peptide or the plurality of peptides comprise, or consist essentially of, or yet further consist of the amino acid LPPIX₁RX₂ (SEQ ID NO: 1), wherein X₁ or X₂ is any amino acid, or an amino acid selected from G, L, M or N, and wherein X₁ and X₂ can be the same or different from each other. Non-limiting examples of such include an isolated peptide comprising a peptide selected from the group of peptides shown in FIG. 3, and optionally FIG. 3A or an isolated peptide comprising or consisting essentially of, or yet further consisting of an amino acid selected from WDLPPIGRLSGN (SEQ ID NO: 2) or THLPPIMRNLQF (SEQ ID NO: 3), SWMPPILRSPAV, SWLPNIQRHWLS, HLPPILRMLDLV, HLPPIQRTPTYA, LPPIVRLPGLLH, FPFGPINRDMTA, or NGVWLPPIARVL joined to a peptide linker, e.g., a linker selected from GGS (SEQ ID NO: 4) or GGSK (SEQ ID NO: 5) or GGGSK. In another aspect of the method, the isolated peptide comprises, or consists essentially of, or yet further consists of the amino acid WDLPPIGRLSGN (SEQ ID NO: 2) that is joined to a peptide linker, e.g., a linker selected from GGS (SEQ ID NO: 4) or GGSK (SEQ ID NO: 5) or GGGSK (SEQ ID NO: 13). In one aspect, the isolated peptide or the plurality of peptides are detectably labeled.

The peptides and compositions are further useful to label a phage present in a sample, comprising contacting the sample with the peptide or plurality of peptides as described herein, and detecting the binding of the peptide to the phage, wherein the presence of binding is a detection of the phage in the sample. The sample can be a clinical isolate or other sample where phages may be present. The sample can be from any species or subject, e.g. a mammal such as a human patient. In one aspect of the above method, the isolated peptide or the plurality of peptides comprise, or consist essentially of, or yet further consist of the amino acid LPPIX₁RX₂ (SEQ ID NO: 1), wherein X₁ or X₂ is any amino acid, or an amino acid selected from G, L, M or N, and wherein X₁ and X₂ can be the same or different from each other that are detectably labeled. Non-limiting examples of such include an isolated peptide comprising a peptide selected from the group of peptides shown in FIG. 3, and optionally FIG. 3A or an isolated peptide comprising or consisting essentially of, or yet further consisting of the amino acid selected from WDLPPIGRLSGN (SEQ ID NO: 2) or THLPPIMRNLQF (SEQ ID NO: 3), SWMPPILRSPAV, SWLPNIQRHWLS, HLPPILRMLDLV, HLPPIQRTPTYA, LPPIVRLPGLLH, FPFGPINRDMTA, or NGVWLPPIARVL joined to a peptide linker, e.g., a linker selected from GGS (SEQ ID NO: 4) or GGSK (SEQ ID NO: 5) or GGGSK, that are detectably labeled. In another aspect, the detectably labeled isolated peptide comprises, or consists essentially of, or yet further consists of the amino acid WDLPPIGRLSGN (SEQ ID NO: 2) that is joined to a peptide linker, e.g., a linker selected from GGS (SEQ ID NO: 4) or GGSK (SEQ ID NO: 5) or GGGSK.

The isolated peptides also are useful to monitor anti-pseudomonas therapy by administering to a subject in need thereof a radiolabeled isolated peptide of this disclosure to a subject in need thereof and detecting the presence of any peptide bound to a GV phage or GV phage infected cell if present in the subject. Any method to detect radiolabels in a subject can be used in combination with this method. In one aspect, the anti-pseudomonas therapy comprises a therapy to treat an GV phage infected cell. In one aspect of the above method, the isolated peptide or the plurality of peptides comprise, or consist essentially of, or yet further consist of the amino acid LPPIX₁RX₂ (SEQ ID NO: 1), wherein X₁ or X₂ is any amino acid, or an amino acid selected from G, L, M or N, and wherein X₁ and X₂ can be the same or different from each other. Non-limiting examples of such include an isolated peptide comprising a peptide selected from the group of peptides shown in FIG. 3, and optionally FIG. 3A or an isolated peptide comprising or consisting essentially of, or yet further consisting of an amino acid selected from WDLPPIGRLSGN (SEQ ID NO: 2) or THLPPIMRNLQF (SEQ ID NO: 3), SWMPPILRSPAV, SWLPNIQRHWLS, HLPPILRMLDLV, HLPPIQRTPTYA, LPPIVRLPGLLH, FPFGPINRDMTA, or NGVWLPPIARVL joined to a peptide linker, e.g., a linker selected from GGS (SEQ ID NO: 4) or GGSK (SEQ ID NO: 5) or GGGSK. In another aspect of the method, the isolated peptide comprises, or consists essentially of, or yet further consists of the amino acid WDLPPIGRLSGN (SEQ ID NO: 2) that is joined to a peptide linker, e.g., a linker selected from GGS (SEQ ID NO: 4) or GGSK (SEQ ID NO: 5) or GGGSK. In one aspect, the isolated peptide or the plurality of peptides are detectably labeled.

The following examples are intended to illustrate but not limit the disclosure.

### Materials and Methods

### Preparation of Good Vibes Phage (GV)

Wastewater from the Tijuana River, San Diego, CA, USA (32°33′24.7″N, 117°07′09.7″W) was centrifuged (4000 g, 10 min, 4° C.) to remove soil particles and other debris. The supernatant was passed through a 0.45-μm filter and added to an overnight P. aeruginosa culture (incubated overnight at 37° C. in lysogeny broth (LB), shaking at 200 rpm). The culture was diluted OD=0.2, which represents the stationary phase. The centrifugation, filtration and inoculation steps were repeated daily for the next 5 days. On the final day, the sample was centrifuged and filtered as above, and 4 μL of the filtrate was spotted onto a P. aeruginosa lawn and incubated at 37° C. overnight to form plaques.

The plaques were picked and suspended in phosphate-buffered saline (PBS) as 10-fold serial dilutions. Applicant plated 100 μL of the 10⁻⁵ and 10⁻⁶ dilutions with 100 μL P. aeruginosa (OD=0.2) and 3-4 mL of warm top agar. At these dilutions, well-separated plaques were able to form in the whole-plate assays, allowing us to distinguish between phage morphologies. Plates were left in the incubator overnight at 37° C. The following day individual plaques were identified, picked and suspended in PBS. The assays were carried out three times to ensure that all morphologies were identical, thus representing homogeneous phages.

The phages were harvested and purified by picking plaques from the whole-plate assays and suspending them in 200 μL PBS. Applicant then mixed 200 μL of an overnight P. aeruginosa culture (OD=0.2) with 25 mL LB and incubated for 20 min at 37° C., shaking at 200 rpm. 25 μL 0.001M MgCl₂, 25 μL 0.001 M CaCl₂) and 200 μL of the phage suspension was added and incubated overnight as above. The next day the culture was centrifuged (4000 g, 30 min, 4° C.) and passed through two 0.45-μm filters to remove bacterial cells before storage at 4° C. Titers of the stock were determined by serial dilution using the whole-plate plaque assay method described above.

### Isolation of GV-Binding Peptides

GV-binding peptides (GVBPs) were isolated using a PhD-12 Phage Display Peptide Library Kit (New England Biolabs) as previously described, with slight modifications. (Chan et al., Biomacromolecules, (2021)) Each well of a Nunc Maxisorp flat-bottom 96-well plate was coated with 10¹⁰ phage per unit (pfu) GV overnight at 4° C. Three rounds of affinity selection were carried out to enrich for GVBPs by increasing the stringency of selection in each round. This was achieved by washing with Tris-buffered saline (TBS) containing increasing concentrations of Tween-20 (TBST) from 0.1% to 0.3% to 0.5%. The enriched phages were eluted and amplified according to the manufacturer's protocol.

### Characterization of GV

GV phages were characterized by transmission electron microscopy (TEM) as previously reported (Chan et al., ACS Nano, (2020)). Briefly, 5 μL of 0.2 mg/mL GV phages was diluted in Milli-Q water and was adsorbed to Formvar/carbon-coated 400 mesh copper grids (Electron Microscopy Science) for 2 min. The grid was washed with 5 μL of water for 1 min followed by adsorption of 5 μL of 2% (w/v) uranyl acetate (Fisher Scientific) for 2 min. Solution was removed from the grid by blotting with filter paper. TEM grids were imaged with FEI Tecnai G2 Spirit transmission microscope at 80 kV.

### Spot Test Assay

Overnight cultures of P. aeruginosa were incubated in fresh 2×yeast extract tryptone (YT) medium at 37° C. until the OD reached ~0.2. Applicant then mixed 200 μL of the culture with 3-5 mL warm soft agar (0.5% w/v) and layered it onto the solid agar plate. The soft agar was allowed to solidify for 15 min under flowing air. Applicant then spotted 10 μL of the phage dilution onto the agar plate and air dried for another 15 min. The plate was then inverted and incubated at 37° C. overnight. The presence of clear zones at the spotting sites was recorded the next day.

### Whole Genome Sequencing of GV Phage

Phage DNA was extracted from 100 μL of high-titer phage lysates using the Qiagen DNeasy Blood and Tissue Kit. DNA quantification was performed and standardized using the Qubit dsDNA HS assay. DNA preparation and sequencing was performed using the Illumina Nextera DNA FlexKit and Adapter Indexes followed by whole genome sequencing using the lab's MiSeq sequencing platform. Sequencing reads were downloaded from Illumina Basespace, then trimmed for length and quality, and assembled de novo using CLC Genomics Workbench 9.

### Polyclonal ELISA

A Nunc Maxisorp flat-bottom 96-well plate was coated with 6×10⁹ pfu GV per well (in TBS, pH 8) and incubated overnight at 4° C. Plates coated with 2% (w/v) bovine serum albumin (BSA) were used as negative controls. Next day, the plates were blocked with 2% (w/v) BSA at room temperature for 1 h, shaking at 800 rpm. The plates were then washed with 0.1% TBST (3×1 min) before adding 20 μL of amplified phage from each biopanning cycle to each well in 5% (w/v) BSA. After further incubation at room temperature for 1 h, shaking at 800 rpm, the plates were washed with 0.5% TBST (3×5 min) before adding 100 μL horseradish peroxidase (HRP)-conjugated anti-M13 monoclonal antibody (Abcam ab50370, diluted 1:500) and incubating at room temperature for 1 h, shaking at 800 rpm. After further washes in 0.5% TBST (3×5 min), Applicant added 100 μL of the tetramethylbenzidine (TMB) substrate (Thermo Fisher Scientific) to each well. The plates were incubated in the dark for 10 min and the absorbance was measured at 370 nm using an Infinite 200 Rx plate reader (Tecan Life Sciences) with 25 flashes in 96-well flat-bottom plate mode.

### Monoclonal ELISA

The protocol was similar to the polyclonal ELISA. Applicant added 100 μL of amplified phage from the previous biopanning cycle to each well, followed by incubation at room temperature for 1 h, shaking at 800 rpm. Monoclonal phages differing in absorbance between GV and BSA by at least 0.3 units were isolated for DNA sanger sequencing (Eurofins Genomics).

### Cross-Reactivity Assay

The protocol was similar to the polyclonal ELISA. Applicant coated wells with 10 μg cowpea mosaic virus (CPMV) or 1010 GV particles to assess their binding activity.

### Synthesis and Testing of GV-Binding Peptides

The linear peptides GVBP-biotin (H₂N-WDLPPIGRLSGNGGGSK/biotin/—CO₂H) and GVBP-FITC (H₂N-WDLPPIGRLSGNGGGSK/Cy5/—CO₂H) were prepared by solid phase peptide synthesis (GenScript) with a purity of 75%. Peptide sequence H₂N-WDLPPIGRLSGN—CO₂H was obtained from the most prevalent monoclonal phages in terms of hits. Applicant added the C-terminal linker GGGS to improve flexibility as well as a lysine residue to provide a side-chain amide bond that could be used to attach cyanine 5 (Cy5) by addition or biotin by substitution.***

### GVBP-Biotin ELISA

GV (1, 10 or 65 μg) was coated onto Nunc Maxisorp flat-bottom 96-well plates and incubated for 1 h at room temperature, shaking at 400 rpm. All incubation and washing steps were carried out at room temperature, shaking at 400 rpm, unless otherwise stated. The wells were blocked for 1 h with 300 μL 5% (w/v) BSA followed by washing once with 0.1% PBST for 5 min. Applicant then added 0.01 μg of GVBP-biotin peptide to the wells and incubated for 1 h. The wells were then washed three times with 0.1% PBST for 5 min each before adding streptavidin HRP conjugate (ab7403) diluted 1:10,000 and incubating for 30 min. The wells were washed another three times as above before adding 100 μL of TMB substrate and incubating for 5-10 min in the dark. The reaction was stopped by adding 50 μL 2M H₂SO₄ and the absorbance was measured at 450 nm using an Infinite 200 Rx plate reader as described above.

### GVBP-Cy5 ELISA

GV (0.1, 1, 10 or 63 μg) was coated onto Corning Costar 96-well white solid plates and incubated for 1 h at room temperature, shaking at 400 rpm. All incubation and washing steps were carried out at room temperature, shaking at 400 rpm, unless otherwise stated. The wells were blocked for 1 h with 300 μL 5% (w/v) BSA followed by washing once with 0.1% PBST for 5 min. Applicant then added 0.05 μg of GVBP-Cy5 peptide and incubated for 1 h. The wells were washed three times with 0.1% PBST for 5 min each before adding 100 μL of distilled water. The fluorescence was measured using an Infinite 200 Rx plate reader with excitation and emission wavelengths of 647 and 665 nm, respectively, and a gain of 50.

### SYBR Tagging of GVER2738 Monoclonal Phage

Applicant mixed 200 μg of the GVER3738 monoclonal phage with 0.5 μL SYBR safe DNA gel stain (APExBIO) and topped up to 500 μL with distilled water. After mixing on a rotator for 15 min at room temperature and passing through a 0.45-μm PES filter to remove unbound SYBR dye, the protein concentration was quantified using the Pierce BCA protein assay kit (Thermo Fisher Scientific). Applicant fractionated 10 μg of GVER2738 with and without SYBR by 1.2% (w/v) TAE agarose gel electrophoresis for 35 min at 110 V to assess the intercalation of the SYBR stain. The same gel was stained with Coomassie Brilliant Blue to show the colocalization of SYBR-stained nucleic acid and phage coat proteins.

### Competitive ELISA

The protocol was similar to the polyclonal ELISA with slight modifications to obtain the KD value. Applicant coated the wells with 5 μg of GV, then GVER2738 (1, 0.5, 1, 2, 3, 4 or 5 μg) was added to the wells together with GVER2738-SYBR (1, 0.5, 1, 2, 3, 4 or 5 μg). Bound GVER2738-SYBR was detected using an Infinite 200 Rx plate reader as described above.

### Flow Cytometry

An overnight culture of P. aeruginosa was diluted 100-fold in fresh medium. The culture was incubated at 37° C. to reach OD₆₀₀~0.2 before adding ~10¹¹ pfu of GV to the cells and incubating at 37° C. for 15 min. The cells were pelleted by centrifugation (6000 g, 15 min, at room temperature), washed once with 800 μL TM buffer (10 mM Tris base, 5 μM CaCl₂), 10 mM MgSO₄, pH 7.4) and resuspended in 200 μL of the same buffer. Applicant added GVBP-Cy5 (2×10¹³ units) and incubated the mixture for 15 min at room temperature before centrifugal washing three times with 0.1% PBST (6000 g, 5 min, at room temperature). The pellet was resuspended in 200 μL TM buffer and the binding of GVBP-Cy5 to GV was analyzed using an Accuri C6 Plus flow cytometer (BD Biosciences). A total of 30,000 events were collected for each analysis.

### Results and Discussion

### Isolation, Preparation, and Characterization of Good Vibes Phage (GV)

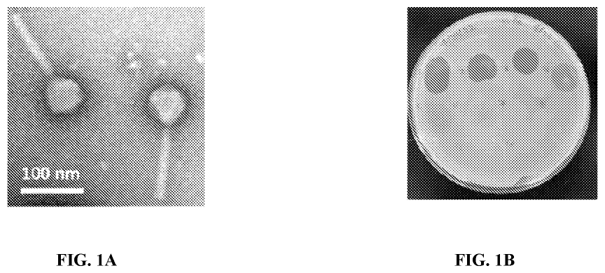

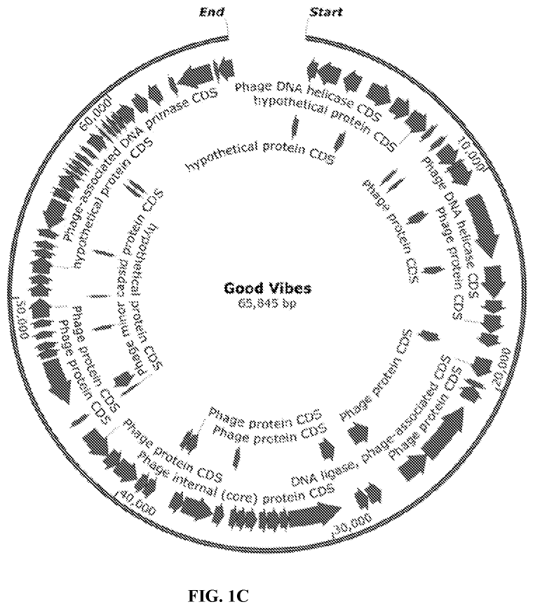

Applicant isolated a lytic phage that infected P. aeruginosa and named it Good Vibes phage (GV). Morphological analysis by transmission electron microscopy (TEM) placed the phage in the family Myoviridae, featuring an icosahedral head, contractile tail and tail fibers connecting the base plate (FIG. 1A). The spot test assay confirmed the lytic activity of GV by producing clear plaques on a bacterial lawn of P. aeruginosa (FIG. 1B). Whole-genome sequencing of GV revealed that the total length of its linear genomic DNA was 65.8 kbp consisting of 24% A, 28.6% C, 23.7% G, and 23.7% T, with 92 predicted coding sequences (FIG. 1C).

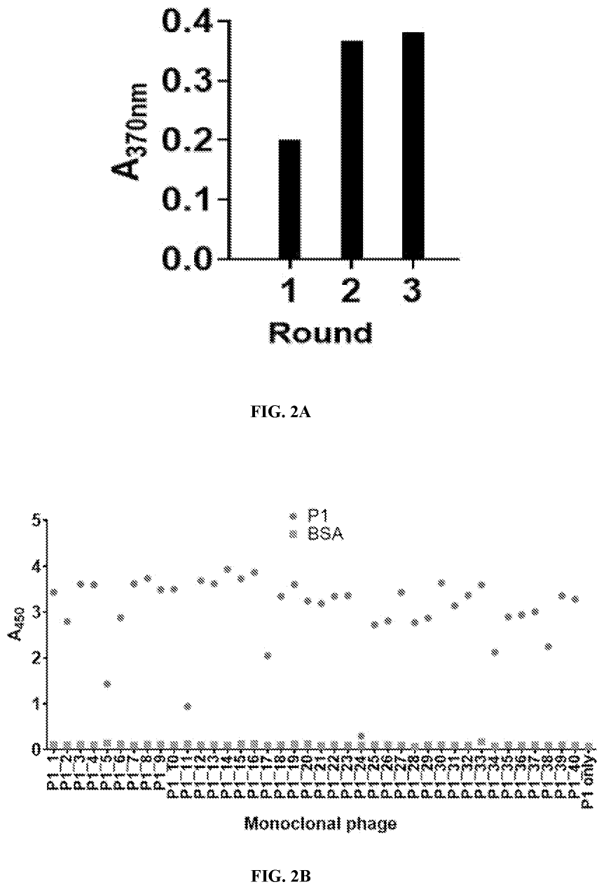

Three rounds of biopanning were carried out with a PhD-12 Phage Display Peptide Library Kit to isolate GV-binding peptides (GVBPs). The stringency was increased in every round to remove non-specific binders and to enrich for monoclonal library phages against GV, which was confirmed by the increasing absorbance signal detected by polyclonal ELISA (FIG. 2A). Forty monoclonal phages from the third round were randomly picked for monoclonal ELISA against the target (GV) and BSA as a negative control (FIG. 2B). Monoclonal library phages with an absorbance difference of 1.0 were selected for Sanger sequencing.

The main advantage of phage display technology is that the genotype (DNA sequence) and the phenotype (displayed peptide) are directly linked, allowing us to determine the nature of the peptide binders by DNA sequencing (Wu et al., J. Biomed. Sci., (2016)). Applicant identified nine unique peptide sequences enriched against GV (FIG. 3A). The most prevalent peptide sequence (WDLPPIGRLSGN) accounted for 50% of the sequenced phages. Applicant also observed the consensus motif LPPI in most of the peptide sequences (FIG. 3A, bolded). Peptide sequence alignment revealed strong conservation, with a consistency score of at least 77 out of 100 (FIG. 3B). The major hit WDLPPIGRLSGN was therefore chosen for synthesis and further characterization.

Cross-reactivity assays, in which the nine unique monoclonal phages were screened against the target (GV), Matera (MAT, also representing the family Myoviridae), cowpea mosaic virus (CPMV, a plant virus), and BSA as a negative control, showed that monoclonal phage displaying peptides WDLPPIGRLSGN or THLPPIMRNLQF produced a strong signal against both GV and MAT, but not against CPMV or BSA (FIG. 3C). Cross-reaction to MAT was anticipated because MAT and GV are closely related and share a high degree of structural similarity. SAROTUP analysis predicted that the WDLPPIGRLSGN peptide is not polystyrene binder and contains no target-unrelated motifs (FIG. 3D). However, monoclonal phages displaying this peptide were predicted to be fast growing, suggesting the phages have a higher infection rate or secretion rate but may not display a target-specific binder. (Thomas et al., Anal. Biochem., (2010)); (Brammer et al., Anal. Biochem., (2008))

### Validation of Monoclonal Phage GVER2738 Binding to GV

The binding of monoclonal phage GVER2738 to GV was confirmed by ELISA. Applicant showed that monoclonal phages displaying the peptide WDLPPIGRLSGN bound more strongly to GV and MAT than the unrelated virus TRI-180 and the negative control BSA (FIG. 4A). GVER2738 also bound more strongly to GV (our target) than the closely related virus MAT. Applicant also compared the binding activity of GVER2738 and empty M13 phage (M13KE). Monoclonal phages displaying peptide WDLPPIGRLSGN bound more strongly than M13KE to GV and MAT (FIG. 4B). Again, GVER2738 also bound more strongly to GV than MAT. These results confirmed that the binding of GVER2738 to GV and MAT reflected the recognition of the displayed peptide rather than the M13 capsid.

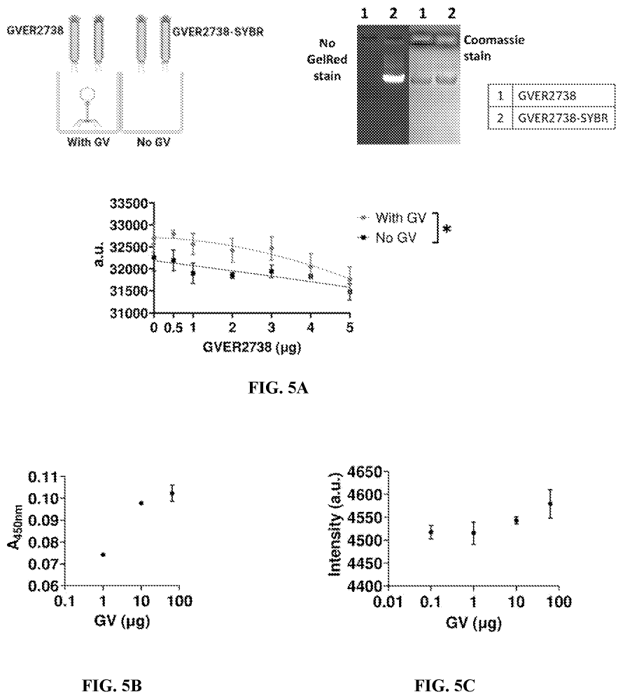

Next, Applicant carried out a competitive binding assay between monoclonal phage GVER2738 and GVER2738 intercalated with SYBR dye (GVER2738_SYBR) with GV as the target (FIG. 5A). The same amount of GV was coated onto the plates and the same amount of GVER2738_SYBR was used in each assay. By increasing the amount of GVER2738, fewer binding sites on the GV surface were available for its competitor GVER2738_SYBR, resulting in a weaker SYBR signal with the increasing amount of GVER2738. The signal from the wells coated with GV was much stronger than that from the negative control wells (coated with BSA), indicating a specific noncovalent interaction between the peptides displayed on the monoclonal phage and GV. The KD value of the binding with GV was ~12 times higher than BSA. To confirm these results, Applicant synthesized the WDLPPIGRLSGN peptide, which Applicant describes as the GV-binding peptide (GVBP), with two separate C-terminal modifications: biotin (GVBP-biotin) and Cy5 (GVBP-Cy5). Applicant confirmed by ELISA that the intensity of signals representing bound GVBP-biotin and GVBP-Cy5 became stronger with increasing amounts of GV coating the plates (FIG. 5B and FIG. 5C).

### Flow Cytometry Analysis of GVBP-Cy5 Binding to GV

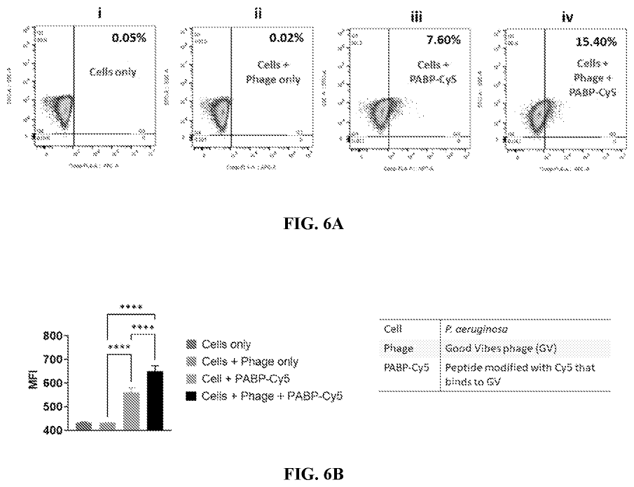

The binding of GVBP-Cy5 to GV in a population of P. aeruginosa cells was investigated by flow cytometry (FIG. 6A). The adherence of GVBP-Cy5 on GV bound to P. aeruginosa cells can be detected by flow cytometry using the APC channel. Higher GVBP-Cy5 bound to GV leads to higher APC shift compared to population without GVBP-Cy5. The population containing both cells and phages (FIG. 6A, panel iv) showed the highest APC+ shift of 15.4% in the presence of GVBP-Cy5, although the population containing cells without phages also showed a moderate shift of 7.6% when the peptide was added, indicating that the peptide binds nonspecifically to the bacterial cells (FIG. 6A, panel iii). These results correlated with the median fluorescence intensity (MFI) values (FIG. 6B). The population containing cells and phages plus GVBP-Cy5 generated the strongest fluorescent signal, indicating the binding of GVBP-Cy5 to phage particles. A weaker signal was detected in the absence of phage. The binding of GVBP-Cy5 to the phage therefore increases the APC+ signal.

Data support that P. aeruginosa cells can be tracked and imaged using the identified GVBP. To proceed with in vivo studies, sensitivity of the approach should be improved; this may be achieved through multivalency therefore introducing avidity effects, e.g., multivalent peptides could be synthesized, and nanoparticle formulations could be utilized to generate high multivalency.

### In Vivo Therapy Monitoring

The present technology is useful to monitor anti-pseudomonas therapy in patients, such as for example, pseudomonas-colonized patients with cystic fibrosis. A cocktail of anti-pseudomonas bacteriophages is administered and pseudomonas infection can be detected in expectorated sputum. Radiolabeled peptide ligands can enable real time visualization of phage populations in the lungs of patients to monitor phage populations at sites of infection. In this application, radiolabeled peptide phage ligand accumulation at sites of infection will be a function of local phage populations which will, in turn, be dependent on bacterial population size. With treatment success, bacterial and, subsequently phage, populations will diminish. This could be of particular value in diagnosing and monitoring patients whose infections are internally localized such as those with prosthetic valve endocarditis, infected pacemakers, prosthetic joints, implanted defibrillators and left ventricular assist devices as well as those with infected synthetic vascular grafts. As phage therapy evolves from environmental to synthetic phages, this imaging approach can be utilized as a companion diagnostic technology that would enable the use of broad host range synthetic bacteriophages to localize infections following parenteral administration of phages and to then image them with radionuclide labeled peptide ligands that would follow them to the sites of infection. Current imaging technologies are dependent on detection of structural disruption or non-specific inflammation. This approach would enable serial imaging of specific pathogens at the site of infection over the course of treatment.

### Experimental Discussion

Applicant successfully isolated 12-mer peptides binding to GV, a phage that infects and lyses the pathogenic bacterium P. aeruginosa. Nine unique peptide sequences were isolated using peptide phage display technology, and the consensus motif LPPI was found in most of the peptides following multiple sequence alignment. ELISAs using monoclonal phage GVER2738 and modified GVBPs confirmed that the peptides bind to GV and closely related phage but not to unrelated viruses. Flow cytometry also showed the significant binding of Cy5-labeled GVBP to GV. Without being bound by theory, the peptides can facilitate the development of in vivo tracers for the real-time analysis of phage therapy.

### EQUIVALENTS

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

[1] K. E. Thorpe, P. Joski, K. J. Johnston, Antibiotic-resistant Infection Treatment Costs Have Doubled Since 2002, Now Exceeding $2 Billion Annually, Health Affairs 37(4) (2018).
[2] H. D. Marston, D. M. Dixon, J. M. Knisely, T. N. Palmore, A. S. Fauci, Antimicrobial Resistance, JAMA 316(11) (2016) 1193-1204.
[3] J. P. Burnham, M. A. Olsen, M. H. Kollef, Re-estimating annual deaths due to multidrug-resistant organism infections, Infect Control Hosp Epidemiol 40(1) (2019) 112-113.
[4] W.H. Organization., Global Action Plan on Antimicrobial Resistance [Internet]., Geneva: WHO. Available from: http://www.wpro.who.int/entity/drug_resistance/resources/global_action_plan_eng.pdf (cited 2019 Apr. 1).
[5] F. L. Gordillo Altamirano, J. J. Barr, Phage Therapy in the Postantibiotic Era, Clin Microbiol Rev 32(2) (2019).
[6] K. E. Kortright, B. K. Chan, J. L. Koff, P. E. Turner, Phage Therapy: A Renewed Approach to Combat Antibiotic-Resistant Bacteria, Cell Host Microbe 25(2) (2019) 219-232.
[7] N. Chanishvili, Phage therapy—history from Twort and d'Herelle through Soviet experience to current approaches, Adv Virus Res 83 (2012) 3-40.
[8] R. T. Schooley, B. Biswas, J. J. Gill, A. Hernandez-Morales, J. Lancaster, L. Lessor, J. J. Barr, S. L. Reed, F. Rohwer, S. Benler, A. M. Segall, R. Taplitz, D. M. Smith, K. Kerr, M. Kumaraswamy, V. Nizet, L. Lin, M. D. McCauley, S. A. Strathdee, C. A. Benson, R. K. Pope, B. M. Leroux, A. C. Picel, A. J. Mateczun, K. E. Cilwa, J. M. Regeimbal, L. A. Estrella, D. M. Wolfe, M. S. Henry, J. Quinones, S. Salka, K.A. Bishop-Lilly, R. Young, T. Hamilton, Development and Use of Personalized Bacteriophage-Based Therapeutic Cocktails To Treat a Patient with a Disseminated Resistant Acinetobacter baumannii Infection, Antimicrob Agents Chemother 61(10) (2017).
[9] S. LaVergne, T. Hamilton, B. Biswas, M. Kumaraswamy, R. T. Schooley, D. Wooten, Phage Therapy for a Multidrug-Resistant Acinetobacter baumannii Craniectomy Site Infection, Open Forum Infect Dis 5(4) (2018) ofy064.
[10] S. Aslam, V. Pretorius, S. M. Lehman, S. Morales, R. T. Schooley, Novel bacteriophage therapy for treatment of left ventricular assist device infection, J Heart Lung Transplant (2019).
[11] R. Nir-Paz, D. Gelman, A. Khouri, B. M. Sisson, J. Fackler, S. Alkalay-Oren, L. Khalifa, A. Rimon, O. Yerushalmy, R. Bader, S. Amit, S. Coppenhagen-Glazer, M. Henry, J. Quinones, F. Malagon, B. Biswas, A. E. Moses, G. Merril, R. T. Schooley, M. J. Brownstein, Y. A. Weil, R. Hazan, Successful treatment of antibiotic resistant poly-microbial bone infection with bacteriophages and antibiotics combination, Clin Infect Dis (2019).
[12] R. M. Dedrick, C. A. Guerrero-Bustamante, R. A. Garlena, D. A. Russel, K. Ford, K. Harris, K. C. Gilmour, J. Soothill, D. Jacobs-Sera, R. T. Schooley, G. F. Hatfull, H. Spencer, Use of engineered bacteriophages for personalized treatment of a patient with a disseminated drug resistant Mycobacterium abscessus infection, Nat Med in press. (2019).
[13] R. Kumar, S. Basu, D. Torigian, V. Anand, H. Zhuang, A. Alavi, Role of modern imaging techniques for diagnosis of infection in the era of 18F-fluorodeoxyglucose positron emission tomography, Clin Microbiol Rev 21(1) (2008) 209-24.
[14] M. Rusckowski, S. Gupta, G. Liu, S. Dou, D. J. Hnatowich, Investigations of a (99m)Tc-labeled bacteriophage as a potential infection-specific imaging agent, J Nucl Med 45(7) (2004) 1201-8.
[15] M. Rusckowski, S. Gupta, G. Liu, S. Dou, D. J. Hnatowich, Investigation of four (99m)Tc-labeled bacteriophages for infection-specific imaging, Nucl Med Biol 35(4) (2008) 433-40.
[16] D. Holman, M. P. Lungren, J. Hardy, C. Contag, F. Blankenberg, Preparation of Tc99m-Labeled Pseudomonas Bacteriophage without Adversely Impacting Infectivity or Biodistribution, Bioconjug Chem 28(11) (2017) 2698-2706.
[17] L. Debarbieux, D. Leduc, D. Maura, E. Morello, A. Criscuolo, O. Grossi, V. Balloy, L. Touqui, Bacteriophages can treat and prevent Pseudomonas aeruginosa lung infections, J Infect Dis 201(7) (2010) 1096-104.
[18] D. R. Roach, C. Y. Leung, M. Henry, E. Morello, D. Singh, J. P. Di Santo, J. S. Weitz, L. Debarbieux, Synergy between the Host Immune System and Bacteriophage Is Essential for Successful Phage Therapy against an Acute Respiratory Pathogen, Cell Host Microbe 22(1) (2017) 38-47 e4.
[19] K. T. O'Neil, R. H. Hoess, S. A. Jackson, N. S. Ramachandran, S. A. Mousa, W. F. DeGrado, Identification of novel peptide antagonists for GPIIb/IIIa from a conformationally constrained phage peptide library, Proteins 14(4) (1992) 509-15.
[20] A. E. Czapar, B. D. B. Tiu, F. A. Veliz, J. K. Pokorski, N. F. Steinmetz, Slow-Release Formulation of Cowpea Mosaic Virus for In Situ Vaccine Delivery to Treat Ovarian Cancer, Adv Sci (Weinh) 5(5) (2018) 1700991.
[21] P. H. Lizotte, A. M. Wen, M. R. Sheen, J. Fields, P. Rojanasopondist, N. F. Steinmetz, S. Fiering, In situ vaccination with cowpea mosaic virus nanoparticles suppresses metastatic cancer, Nat Nanotechnol 11(3) (2016) 295-303.
[22] M. Bruckman, K. Jiang, E. J. Simpson, L. Randolph, L. G. Luyt, X. Yu, N. F. Steinmetz, Dual-modal magnetic resonance and fluorescence imaging of atherosclerotic plaques in vivo using VCAM-1 targeted tobacco mosaic virus, Nano Lett 14(3) (2014) 1551-1558.
[23] H. Hu, Y. Zhang, S. Shukla, Y. Gu, X. Yu, N. F. Steinmetz, Dysprosium-Modified Tobacco Mosaic Virus Nanoparticles for Ultra-High-Field Magnetic Resonance and Near-Infrared Fluorescence Imaging of Prostate Cancer, ACS Nano 11(9) (2017) 9249-9258.
[24] H. S. Leong, N. F. Steinmetz, A. Ablack, G. Destito, A. Zijlstra, H. Stuhlmann, M. Manchester, J.D. Lewis, Intravital imaging of embryonic and tumor neovasculature using viral nanoparticles, Nat Protoc 5(8) (2010) 140617.
[25] C. F. Cho, L. Yu, T. K. Nsiama, A. N. Kadam, A. Raturi, S. Shukla, G. A. Amadei, N. F. Steinmetz, L. G. Luyt, J. D. Lewis, Viral nanoparticles decorated with novel EGFL7 ligands enable intravital imaging of tumor neovasculature, Nanoscale 9(33) (2017) 12096-12109.
[26] P. V. Chang, J. A. Prescher, E. M. Sletten, J. M. Baskin, I. A. Miller, N. J. Agard, A. Lo, C.R. Bertozzi, Copper-free click chemistry in living animals, Proc Natl Acad Sci USA 107(5) (2010) 1821-6.
[27] V. Hong, N. F. Steinmetz, M. Manchester, M. G. Finn, Labeling live cells by copper-catalyzed alkyne-azide click chemistry, Bioconjug Chem 21(10) (2010) 1912-6.

1. An isolated peptide comprising the amino acid LPPIX1RX2 (SEQ ID NO: 1), wherein X₁ or X₂ is any amino acid, or an amino acid selected from G, L, M or N, and wherein X₁ and X₂ can be the same or different from each other.

2. An isolated peptide comprising a peptide selected from the group of peptides shown in FIG. 3, and optionally FIG. 3A.

3. An isolated peptide comprising the amino acid selected from an amino acid from the group of WDLPPIGRLSGN (SEQ ID NO: 2) or THLPPIMRNLQF (SEQ ID NO: 3), SWMPPILRSPAV (SEQ ID NO: 6), SWLPNIQRHWLS (SEQ ID NO: 7), HLPPILRMLDLV (SEQ ID NO: 8), HLPPIQRTPTYA (SEQ ID NO: 9), LPPIVRLPGLLH (SEQ ID NO: 10), FPFGPINRDMTA (SEQ ID NO: 11), or NGVWLPPIARVL (SEQ ID NO: 12).

4. An isolated peptide of claim 1, further comprising a linker.

5. The isolated peptide of claim 4, wherein the linker comprises GGS, GGSK (SEQ ID NO: 5), or GGGSK (SEQ ID NO: 13).

6. The isolated peptide of claim 1, further comprising a detectable label.

7. The isolated peptide of claim 6, wherein the detectable label is selected from a radionucleotide, biotin or cyanine 5 (Cy5).

8. A plurality of isolated peptides of claim 1, that can be the same or different from each other.

9. The isolated peptide of claim 1, bound to a phage or its progeny.

10. The isolated peptide of claim 9, wherein the phage is a Good Vibes phage.

11. A polynucleotide encoding the isolated peptide of claim 1, wherein the polynucleotide is DNA or RNA.

12. A vector or host cell comprising the isolated peptide of claim 1.

13. A composition comprising a carrier and the isolated peptide of claim 1.

14. A method to detect a phage in a sample, comprising contacting the sample with the peptide of claim 1.

15. A method to label a phage in a sample, comprising contacting the sample with the peptide of claim 1.

16. The method of claim 14, wherein the contacting is in vitro or in vivo.

17. The method of claim 14, wherein the sample comprises a clinical isolate.

18. The method of claim 17, wherein the clinical isolate is a mammalian or human clinical isolate.
